SEQUENCE LISTING (9 sequences)

SEQ ID NO: 1 - protein, 618 aa, Borrelia burgdorferi
MKSHILYKLIIFLTTSAAIFAADALKEKDIFKINPWMPTFGFENTSEFRLDMDELVPGFENKSKITIKLKPFEANPELGKDDPFSAYIKVEDLALKAEGKKGDQFKIDVGDITAQINMYDFFIKISTMTDFDFNKESLFSFAPMTGFKSTYYGFPSNDRAVRGTILARGTSKNIGTIQLGYKLPKLDLTFAIGGTGTGNRNQENDKDTPYNKTYQGILYGIQATWKPIKNLLDQNEDTKSVIAETPFELNFGLSGAYGNETFNNSSITYSLKDKSVVGNDLLSPTLSNSAILASFGAKYKLGLTKINDKNTYLILQMGTDFGIDPFASDFSIFGHISKAANFKKETPSDPNKKAEIFDPNGNALNFSKNTELGIAFSTGASIGFAWNKDTGEKESWAIKGSDSYSTRLFGEQDKKSGVALGISYGQNLYRSKDTEKRLKTISENAFQSLNVEISSYEDNKKGIINGLGWITSIGLYDILRQKSVENYPTTISSTTENNQTEQSSTSTKTTTPNLTFEDAMKLGLALYLDYAIPIASISTEAYVVPYIGAYILGPSNKLSSDATKIYLKTGLSLEKLIRFTTISLGWDSNNIIELANKNTNNAAIGSAFLQFKIAYSGS

SEQ ID NO: 2 - protein, 132 aa, Artificial sequence
  synthetic sequence
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARRGNQKDIVVVPAAIFSGENAFDIWGQGTMVTVSS

SEQ ID NO: 3 - protein, 107 aa, Artificial sequence
  synthetic sequence
DIQMTQSPSSLSASVGDRVTITCQASQDISNYLNWYQQKPGKAPKLLIYDASNLETGVPSRFSGSGSGTDFTFTISSLQPEDIATYYCQQYDNLPYTFGQGTKLEIK

SEQ ID NO: 4 - protein, 6 aa, Artificial sequence
  synthetic sequence
SSNWWS

SEQ ID NO: 5 - protein, 16 aa, Artificial sequence
  synthetic sequence
EIYHSGSTNYNPSLKS

SEQ ID NO: 6 - protein, 23 aa, Artificial sequence
  synthetic sequence
RGNQKDIVVVPAAIFSGENAFDI

SEQ ID NO: 7 - protein, 11 aa, Artificial sequence
  synthetic sequence
QASQDISNYLN

SEQ ID NO: 8 - protein, 7 aa, Artificial sequence
  synthetic sequence
DASNLET

SEQ ID NO: 9 - protein, 9 aa, Artificial sequence
  synthetic sequence
QQYDNLPYT

(19) United States
(12) Patent Application Publication
(10) Pub. No.: US 2023/0391857 A1
(43) Pub. Date: Dec. 7, 2023

(54) METHODS OF TREATING INFECTIONS BY BLOCKING PATHOGEN MIMICS OF CD47

(71) Applicant: The Board of Trustees of the Leland Stanford Junior University, Stanford, CA (US)

(72) Inventors: Michal Caspi Tal, Cupertino, CA (US); Balyn Zaro, Redwood City, CA (US); Irving L. Weissman, Stanford, CA (US); Nitya Ramadoss, Redwood City, CA (US); William H. Robinson, Palo Alto, CA (US)

(21) Appl. No.: 18/034,026

(22) PCT Filed: Oct. 29, 2021
(86) PCT No.: PCT/US2021/057286

§ 371 Date: Apr. 26, 2023

Related U.S. Application Data
(60) Provisional application No. 63/107,295, filed on Oct. 29, 2020.

(51) Int. Cl. C07K 16/12; A61K 38/16; A61P 31/04

(52) U.S. Cl. CPC C07K 16/1207; A61K 38/164; A61P 31/04; C07K 2317/24; C07K 2317/21

(57) ABSTRACT

The CD47 mimic protein of Borrelia burgdorferi is identified as p66 protein. Blocking the p66 protein on the pathogen Improves the immune response of an infected individual, and allows a more complete and/or more rapid resolution of Borrelia infection. The disclosure provides a method of inhibiting or treating an infection by a Borrelia pathogen, the method comprising administering an effective amount of an anti-p66 agent that reduces binding of p66 on the pathogen to signal regulatory protein a (SIRPa) on a phagocytic cell, where the agent (i) specifically binds to p66, or (ii) is a p66 polypeptide or derivative thereof.

### CROSS REFERENCE TO RELATED APPLICATION

The present application claims the benefit of and priority to U.S. Provisional Patent Application No. 63/107,295, filed Oct. 29, 2020, the entire disclosure of which is hereby.

### INCORPORATION BY REFERENCE OF SEQUENCE LISTING PROVIDED AS A TEXT FILE

A Sequence Listing is provided herewith in a text file, (STAN-1806WO_SEQ_LIST_ST25.txt), created on Sep. 27, 2021, and having a size of 10000 bytes. The contents of the text file are incorporated herein by reference in its entirety.

### BACKGROUND OF THE INVENTION

Both cancer cells and persistently-infected cells can express a variety of inhibitory ligands that dampen innate and adaptive branches of the immune system. In the case of innate immunity, phagocytic macrophages are responsible for the digestion and degradation of exhausted or foreign cells, viruses and bacteria. Macrophage-mediated programmed cell removal is governed by protein-protein interactions between target cells expressing pro-phagocytic and inhibitory ligands and their cognate receptors on the surface of phagocytic macrophages. These ligand-receptor interactions are often referred to as “don't eat me” signals. To date, four “don't eat me” signals have been identified. CD47 has been identified as a dominant “don't eat me” signal expressed by target cells for macrophages. CD47 is a cell-surface molecule that is highly expressed on healthy red blood cells and upregulated on cancer cells, which inhibit phagocytosis by binding to SIRPα on macrophages. While CD47 is well conserved across humans, SIRPα is highly polymorphic. It has been postulated that this heterogeneity is due to pressure imposed by pathogens on the innate immune response.

Programmed cell death (PCD) and phagocytic cell removal are common ways that damaged, precancerous, inflamed, or infected cells respond to pathogenic threats to the organism. However, some infections persist for long periods of time, suggesting that successful persistent infections overcome the PCD and phagocytic cell removal pathways.

There remains a need for better methods of treating infections to overcome pathogen avoidance of innate immune responses.

### SUMMARY OF THE INVENTION

Compositions and methods are provided for blocking undesirable interactions between Borrelia burgdorferi, and phagocytic cells. Borrelia burgdorferi expresses a CD47 mimic protein that interactions with host SIRPα protein, which is expressed on phagocytic cells, where the interaction inhibits phagocytosis of the pathogen. The CD47 mimic is identified herein as “p66”, (SEQ ID NO:1). Blocking the p66 protein on the pathogen improves the immune response of an infected individual, and allows a more complete and/or more rapid resolution of Borrelia infection.

In some embodiments, a blocking agent that interferes with the interaction between p66 present on a bacterial cell, e.g. a Borellia pathogen, including Borrelia burgdorferi, Borrelia hermsii, Borrelia miyamotoi, Borrelia afzelli, Borrelia garinii, Borrelia turicatae; and SIRPα present on a mammalian cell, e.g. a monocyte, macrophage, dendritic cell, etc. is administered to an infected individual. In some embodiments, the blocking agent increases phagocytosis of the bacterial cell by the mammalian cell.

In some embodiments a p66 blocking agent specifically binds to p66 (SEQ ID NO:1). In some such embodiments, the agent that specifically binds to p66 is an antibody, including without limitation chimeric, humanized, affinity enhanced, etc. antibodies with any mammalian Fc region sequence. In other embodiments, a p66 blocking agent comprises a p66 polypeptide sequence, e.g. a soluble portion of the p66 protein, e.g. the extracellular domain of the protein. In some such embodiments, the p66 protein is a derivative of the p66 protein that is modified by one or more amino acid substitutions or deletions to increase affinity of the protein for binding to SIRPα. The p66 polypeptide may be substantially similar to the soluble portion of SEQ ID NO:1, e.g. at least about 80% sequence identity, at least about 85%, at least about 90%, at least about 95%, at least about 97%, at least about 98%, at least about 99%, or 100% identical.

In some embodiments, an anti-p66 antibody is provided. The antibody may comprise as a variable region binding sequence the CDR sequences of SEQ ID NO:2 and SEQ ID NO:3, or a variant thereof, e.g. an affinity-matured variant. The antibody may comprise as a variable region binding sequence SEQ ID NO:2 and SEQ ID NO:3, or a variant thereof, e.g. an affinity matured variant. The variable region may be formatted in any convenient antibody or CAR-T format. The set of CDR sequences or the variable region sequences may be substantially similar to those of SEQ ID NO:2 and/or SEQ ID NO:3, e.g. at least about 80% sequence identity, at least about 85%, at least about 90%, at least about 95%, at least about 97%, at least about 98%, at least about 99%, or 100% identical.

In some embodiments, a method of inhibiting an infection of a subject by a Borrelia pathogen is provided, the method comprising administering to the subject an effective amount of an agent that reduces binding of p66 on the pathogen to a signal regulatory protein a (SIRPα) on a phagocytic cell, where the agent (i) specifically binds to p66, or (ii) is a p66 polypeptide or derivative thereof. In some embodiments, the p66 polypeptide or the agent that specifically binds to p66 comprises an immunoglobulin Fc domain including, without limitation, IgG1, IgG2, IgG3, IgG4, IgA, IgE, IgD, and IgM Fc sequences. In some embodiments, the subject that is treated by the methods described herein is a mammalian subject, including without limitation, a human, dog, cat, pig, sheep, cow, goat, horse, non-human primate, etc.

In some embodiments, methods provided are used for targeting or depleting a Borrelia pathogen, comprising contacting an infected biological sample, e.g. blood from an infected subject, with an agent that specifically binds to p66 protein in order to target or deplete the pathogen. In certain aspects, the agent is an antibody specific for the p66 protein, optionally conjugated to an antimicrobial agent, antifungal agent, or cytotoxic agent, e.g., radioactive isotope, chemotherapeutic agent, toxin, etc., or a detectable label including, without limitation, a fluorophore, a chemiluminescent label, a bioluminescent label, an isotopic label, or a contrast agent.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. B. burgdorferi express a mimic of CD47, p66, and treatment with a CD47 blocking promotes clearance of infection in vivo. A. B. burgdorferi stain for the high-affinity CD47 blocking reagent CV1-G4 and to a lesser extent the anti-CD47 antibody clone B6H12 by flow cytometry analysis and microscopy. B. IgM binding is decreased in an in vivo model of B. burgdorferi infection when bacteria are pre-treated with CV1-G4 compared to isotype control or no treatment. C. Mass spectrometry analysis identifies p66 as a CV1-G4 binding protein. B. burgdorferi was cultured under conditions to stain either positively or negatively for CV1-G4 as confirmed by flow cytometry. Bacteria was lysed under non-denaturing conditions, and lysate was subjected to enrichment with CV1-G4 or IgG4 prior to SDS-PAGE. Gel bands of interest were excised and subjected to in-gel trypsin digestion followed by mass spectrometry analysis. Data filtration parameters identified p66 as a putative CV1-G4 binding protein.

FIG. 2. p66 is required for CV1-G4 binding and also binds SIRPα. A. Wild-type (WT) or p66 knockout (KO) B. burgdorferi were stained with either CV1-G4 or the anti-CD47 blocking antibody MIAP410, revealing that p66 KO bacteria no longer bind CV1-G4 by flow cytometry. B. Recombinant p66 is enriched by CV1-G4 or SIRPα but not isotype control as determined by in vitro binding assay. A representative blot is shown as is quantification from 4 replicates.

FIG. 3. p66 KO B. burgdorferi are more-readily phagocytosed my human macrophages compared to WT. A. Phagocytosis assays across 4 macrophage donors reveal that p66 KO bacteria phagocytosed better than WT. B. Individual macrophage donor phagocytosis data.

FIG. 4. p66 elicits an immune response in Return to Health (RTH) individuals. B-cells from Post-Treatment Lyme Disease Syndrome (PTLDS) and RTH patients reveal a p66-specific response (pink dot) is elevated in RTH individuals whereas a VIsEl response is found across all patients.

### DETAILED DESCRIPTION OF THE INVENTION

The present invention relates to methods of treating a subject for an infection by administering an agent that reduces the binding of a p66 protein on a pathogen to SIRPα on a phagocytic cell, which may be referred to herein as an anti-p66 agent.

The terms “treatment”, “treating”, “treat” and the like are used herein to generally refer to obtaining a desired pharmacologic and/or physiologic effect. The effect can be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom. Those in need of treatment include those already with an infection as well as those in which an infection is to be prevented. As such, a therapeutic treatment is one in which the subject is infected prior to administration and a prophylactic treatment is one in which the subject is not infected prior to administration. In some embodiments, the subject is suspected of being infected prior to administration. In some embodiments, the subject has an increased risk of infection prior to administration. In some embodiments, the subject is suspected of being at increased risk of infection prior to administration.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

An “effective amount” is an amount sufficient to effect beneficial or desired clinical results in treatment of an infection. By “effective amount” is intended an amount of an anti-p66 agent that is sufficient to palliate, ameliorate, stabilize, reverse, prevent, slow or delay the progression of a disease state (e.g., infection) by increasing phagocytosis of a pathogen expressing a pathogenic p66 protein. An effective amount can be administered in one or more administrations.

As used herein, a “target pathogen” is a pathogen expressing p66 protein on its surface, e.g. Borrelia burgdorferi. Administration of an anti-p66 agent that reduces the binding of a p66 protein on a pathogen to SIRPα on a phagocytic cell results in increased phagocytosis of the target pathogen. The target pathogen may include, but is not limited to bacteria, viruses, protozoans, and fungi that express a p66 protein.

As used herein, the term “anti-p66 agent” refers to an agent that reduces the binding of a p66 protein (e.g., on an infectious pathogen) to SIRPα (e.g., on a phagocytic cell), where the agent (i) specifically binds to p66, or (ii) is a p66 polypeptide or derivative thereof. In some embodiments, a suitable anti-p66 agent, e.g. an antibody specific for the anti-p66, binds to a p66 protein to reduce the binding of the p66 protein to SIRPα. In some embodiments, a suitable anti-p66 agent is a soluble fragment of p66. A suitable anti-p66 agent does not activate SIRPα, e.g., in the SIRPα-expressing phagocytic cell.

The efficacy of a suitable anti-p66 agent can be assessed by assaying the agent. In an exemplary assay, a pathogen comprising a pathogen p66 protein is incubated in the presence or absence of the candidate agent and a phagocytic cell. An agent for use in the methods of the invention will up-regulate phagocytosis by at least 10% (e.g., at least 20%, at least 30%, at least 40%, at least 50%, at least 60%, at least 70%, at least 80%, at least 90%, at least 100%, at least 120%, at least 140%, at least 160%, at least 160%, or at least 200%) compared to phagocytosis in the absence of the agent. Similarly, an in vitro assay for levels of tyrosine phosphorylation of SIRPα will show a decrease in phosphorylation by at least 5% (e.g., at least 10%, at least 15%, at least 20%, at least 30%, at least 40%, at least 50%, at least 60%, at least 70%, at least 80%, at least 90%, or 100%) compared to phosphorylation observed in absence of the candidate agent.

In some embodiments, the anti-p66 agent does not cross-react with human CD47, i.e. it does not bind to or activate CD47 in a mammalian host or cell. When CD47 is activated, a process akin to apoptosis (i.e., programmed cell death) occurs (Manna and Frazier, Cancer Research, 64, 1026-1036, Feb. 1, 2004).

The terms “specific binding,” “specifically binds,” and the like, refer to non-covalent or covalent preferential binding to a molecule relative to other molecules or moieties in a solution or reaction mixture, e.g., an antibody specifically binds to a particular polypeptide or epitope relative to other available polypeptides. In some embodiments, the affinity of one molecule for another molecule to which it specifically binds is characterized by a K_{D} (dissociation constant) of 10⁻⁵ M or less, e.g., 10⁻⁶ M or less, 10⁻⁷ M or less, 10⁻⁸ M or less, 10⁻⁸ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less. “Affinity” refers to the strength of binding, increased binding affinity being correlated with a lower KD. In an embodiment, affinity is determined by surface plasmon resonance (SPR), e.g. as used by Biacore systems. The affinity of one molecule for another molecule is determined by measuring the binding kinetics of the interaction, e.g. at 25° C.

The term “specific binding member” as used herein refers to a member of a specific binding pair (i.e., two molecules, usually two different molecules, where one of the molecules, e.g., a first specific binding member, through non-covalent means specifically binds to the other molecule, e.g., a second specific binding member). Suitable specific binding members include agents that specifically bind to a p66 protein, i.e., anti-p66 agents, or that otherwise block the interaction between a p66 protein and SIRPα.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “antibody” encompasses polyclonal and monoclonal antibody preparations, as well as preparations including hybrid antibodies, altered antibodies, chimeric antibodies and, humanized antibodies, as well as: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, e.g., Huston et al. (1988) Proc Natl Acad Sci USA 85:5879-5883); nanobodies or single-domain antibodies (sdAb) (see, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule. Antibodies may be of an IgG type, comprising an Fc region from any one of IgG1, IgG2a, IgG2b, IgG3, IgG4.

In one embodiment, the anti-p66 agent, or a pharmaceutical composition comprising the agent, is provided in an amount effective to detectably inhibit the binding of p66 protein on a pathogen to SIRPα present on the surface of phagocytic cells. The effective amount is determined via empirical testing routine in the art, for example in a biological sample taken from an infected individual. The effective amount may vary depending on the number of cells being targeted, the location of the cells, and factors specific to the subject.

The terms “phagocytic cells” and “phagocytes” are used interchangeably herein to refer to a cell that is capable of phagocytosis. There are three main categories of phagocytes: macrophages, mononuclear cells (histiocytes and monocytes); polymorphonuclear leukocytes (neutrophils) and dendritic cells.

The term “sample” with respect to a patient encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as cellular pathogens or infected cells. The definition also includes sample that have been enriched for particular types of molecules, e.g., nucleic acids, polypeptides, etc.

The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes an infected sample obtained from a patient infected with a pathogen comprising a pathogenic p66 protein, e.g., a sample comprising the pathogen or cells infected with the pathogen or polynucleotides and/or polypeptides that are obtained from a patient's infected cell (e.g., a cell lysate or other cell extract comprising polynucleotides and/or polypeptides); and a sample comprising pathogens comprising a p66 protein. A biological sample comprising a pathogen or an infected cell from a patient can also include non-infected cells.

p66 polypeptide In some embodiments, a subject anti-p66 agent is a p66-derived polypeptide or analogs thereof. In some embodiments, a p66-derived polypeptide is soluble, where the polypeptide lacks the p66 transmembrane domain, e.g. from about residue 143 to about residue 384 of SEQ ID NO:1. The polypeptide may comprise at least one amino acid change relative to the wild-type sequence, wherein the amino acid change increases the affinity to SIRPα, for example by decreasing the off-rate by at least 10-fold, at least 20-fold, at least 50-fold, at least 100-fold, at least 500-fold, or more. A suitable p66 polypeptide reduces (e.g., blocks, prevents, etc.) the interaction between SIRPα and p66 protein. Such a polypeptide optionally comprises additional amino acid sequences, for example antibody Fc sequences; and the like. The polypeptides may be monomeric or multimeric, i.e. dimer, trimer, tetramer, etc., e.g. multimerized through antibody Fc region sequences.

Anti-p66 antibodies. In some embodiments, a subject anti-p66 agent is an antibody that specifically binds the p66 protein (i.e., an anti-p66 antibody) and reduces the interaction between the p66 protein on a pathogen and SIRPα on another cell (e.g., a phagocytic cell).

The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. “Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the light chain variable domain, without an associated heavy chain moiety (3) single chain polypeptides containing only one heavy chain variable region, or a fragment thereof containing the three CDRs of the heavy chain variable region, without an associated light chain moiety and (4) nanobodies comprising single Ig domains from non-human species or other specific single-domain binding modules; and multispecific or multivalent structures formed from antibody fragments. In an antibody fragment comprising one or more heavy chains, the heavy chain(s) can contain any constant domain sequence (e.g. CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

As used in this invention, the term “epitope” means any antigenic determinant on an antigen to which the paratope of an antibody binds. Epitopic determinants usually consist of chemically active surface groupings of molecules such as amino acids or sugar side chains and usually have specific three-dimensional structural characteristics, as well as specific charge characteristics.

Suitable anti-p66 antibodies include fully human, humanized or chimeric versions of such antibodies. Humanized antibodies are especially useful for in vivo applications in humans due to their low antigenicity. Similarly caninized, felinized, etc. antibodies are especially useful for applications in dogs, cats, and other species respectively.

### Methods

Methods are provided for treating or reducing Borrelia infection by inhibiting the interaction between SIRPα and p66 protein on a Borrelia pathogen, thereby increasing in vivo phagocytosis of the pathogen. Such methods include administering to a subject in need of treatment a therapeutically effective amount or an effective dose of an anti-p66 agent, including without limitation combinations of the anti-p66 agent with another drug.

In some embodiments the infection is a chronic infection, i.e. an infection that is not cleared by the host immune system within a period of up to 1 week, 2 weeks, etc. In some embodiments, the chronic infection is caused by the ability of the Borrelia pathogen comprising the p66 protein to evade the immune system by inhibiting phagocytosis.

Bacterial pathogens of interest include without limitation, Borrelia pathogens that cause human disease such as Borrelia burgdorferi, Borrelia hermsii, Borrelia miyamotoi, Borrelia afzelli, Borrelia garinii, Borrelia turicatae.

The methods of the invention provide for a more effective removal of pathogens comprising a p66 protein on their surface by phagocytic cells of the host organism, relative to phagocytosis in the absence of treatment. In some embodiments, the methods of the invention involve diagnosis of a patient as suffering from an infection by a Borrelia pathogen comprising a p66 protein; or selection of a patient previously diagnosed as suffering from an infection by a Borrelia pathogen comprising a p66 protein; treating the patient with a regimen of anti-p66 therapy, optionally in combination with an additional therapy; and monitoring the patient for efficacy of treatment. Monitoring may measure clinical indicia of infection, e.g. fever, white blood cell count, etc., and/or direct monitoring for presence of the pathogen.

Treatment may be combined with other active agents. Classes of antibiotics include penicillins, e.g. penicillin G, penicillin V, methicillin, oxacillin, carbenicillin, nafcillin, ampicillin, etc.; penicillins in combination with β-lactamase inhibitors, cephalosporins, e.g. cefaclor, cefazolin, cefuroxime, moxalactam, etc.; carbapenems; monobactams; aminoglycosides; tetracyclines; macrolides; lincomycins; polymyxins; sulfonamides; quinolones; cloramphenical; metronidazole; spectinomycin; trimethoprim; vancomycin; etc. Cytokines may also be included, e.g. interferon γ, tumor necrosis factor α, interleukin 12, etc. Antiviral agents, e.g. acyclovir, gancyclovir, etc., may also be used in treatment. Steroids may also be used in treatment.

Effective doses of the therapeutic entity of the present invention vary depending upon many different factors, including the nature of the anti-p66 agent, means of administration, target site, physiological state of the patient, whether the patient is human or an animal, other medications administered, and whether treatment is prophylactic or therapeutic. Usually, the patient is a human, but nonhuman mammals may also be treated, e.g. companion animals such as dogs, cats, horses, etc., laboratory mammals such as rabbits, mice, rats, etc., and the like. Treatment dosages can be titrated to optimize safety and efficacy.

In some embodiments, the therapeutic dosage can range from about 0.0001 to 500 mg/kg, and more usually 0.01 to 100 mg/kg, of the host body weight. For example dosages can be 1 mg/kg body weight or 10 mg/kg body weight or within the range of 1-50 mg/kg, e.g.

A “therapeutically effective dose” or “therapeutic dose” is an amount sufficient to effect desired clinical results (i.e., achieve therapeutic efficacy). A therapeutically effective dose can be administered in one or more administrations. For purposes of this invention, a therapeutically effective dose of an anti-p66 agent is an amount that is sufficient to palliate, ameliorate, stabilize, reverse, prevent, slow or delay the progression of the disease state.

In some embodiments, a therapeutically effective dose leads to sustained serum levels of anti-p66 agent of about 40 μg/ml or more (e.g, about 50 ug/ml or more, about 60 ug/ml or more, about 75 ug/ml or more, about 100 ug/ml or more, about 125 ug/ml or more, or about 150 ug/ml or more). In some embodiments, a therapeutically effective dose leads to sustained serum levels of anti-p66 agent that range from about 40 μg/ml to about 300 ug/ml (e.g, from about 40 ug/ml to about 250 ug/ml, from about 40 ug/ml to about 200 ug/ml, from about 40 ug/ml to about 150 ug/ml, from about 40 ug/ml to about 100 ug/ml, from about 50 ug/ml to about 300 ug/ml, from about 50 ug/ml to about 250 ug/ml, from about 50 ug/ml to about 200 ug/ml, from about 50 ug/ml to about 150 ug/ml, from about 75 ug/ml to about 300 ug/ml from about 75 ug/ml to about 250 ug/ml, from about 75 ug/ml to about 200 ug/ml, from about 75 ug/ml to about 150 ug/ml, from about 100 ug/ml to about 300 ug/ml, from about 100 ug/ml to about 250 ug/ml, or from about 100 ug/ml to about 200 ug/ml). In some embodiments, a therapeutically effective dose for treating solid tumors leads to sustained serum levels of anti p66 agent of about 100 μg/ml or more (e.g., sustained serum levels that range from about 100 ug/ml to about 200 ug/ml).

Accordingly, a single therapeutically effective dose or a series of therapeutically effective doses would be able to achieve and maintain a serum level of anti-p66 agent. A therapeutically effective dose of an anti-p66 agent can depend on the specific agent used, but is may be about 8 mg/kg body weight or more (e.g., about 8 mg/kg or more, about 10 mg/kg or more, about 15 mg/kg or more, about 20 mg/kg or more, about 25 mg/kg or more, about 30 mg/kg or more, about mg/kg or more, or about 40 mg/kg or more), or from about 10 mg/kg to about 40 mg/kg (e.g., from about 10 mg/kg to about 35 mg/kg, or from about 10 mg/kg to about 30 mg/kg). The dose required to achieve and/or maintain a particular serum level is proportional to the amount of time between doses and inversely proportional to the number of doses administered. Thus, as the frequency of dosing increases, the required dose decreases. The optimization of dosing strategies will be readily understood and practiced by one of ordinary skill in the art.

The dosage may be adjusted for the molecular weight of the reagent. An exemplary treatment regime entails administration daily, semi-weekly, weekly, once every two weeks, once a month, etc. In another example, treatment can be given as a continuous infusion. Therapeutic entities of the present invention are usually administered on multiple occasions. Intervals between single dosages can be weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the polypeptide in the patient. It will be understood by one of skill in the art that such guidelines will be adjusted for the molecular weight of the active agent, e.g. in the use of antibody fragments, in the use of antibody conjugates, in the use of high affinity p66 reagents, etc. The dosage may also be varied for localized administration, e.g. intranasal, inhalation, etc., or for systemic administration, e.g. i.m., i.p., i.v., and the like.

For the treatment of disease, the appropriate dosage of the anti-p66 agent will depend on the type of disease to be treated, as defined above, the severity and course of the disease, whether the agent is administered for preventive purposes, previous therapy, the patient's clinical history and response to the antibody, and the discretion of the attending physician. The anti-p66 agent is suitably administered to the patient at one time or over a series of treatments.

Suitable anti-p66 agents can be provided in pharmaceutical compositions suitable for therapeutic use, e.g. for human treatment. In some embodiments, pharmaceutical compositions of the present invention include one or more therapeutic entities of the present invention or pharmaceutically acceptable salts, esters or solvates thereof. In some other embodiments, the use of an anti-p66 agent includes use in combination with another therapeutic agent, e.g., another anti-infection agent. Therapeutic formulations comprising one or more anti-p66 agents of the invention are prepared for storage by mixing the anti-p66 agent having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. The anti-p66 agent composition will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the anti-p66 agent to be administered will be governed by such considerations, and is the minimum amount necessary to prevent the associated disease.

The anti-p66 agent can be administered by any suitable means, including topical, oral, parenteral, subcutaneous, intraperitoneal, intrapulmonary, and intranasal. Parenteral infusions include intramuscular, intravenous, intraarterial, intraperitoneal, intrathecal or subcutaneous administration. In addition, the anti-p66 agent is suitably administered by pulse infusion, particularly with declining doses of the agent.

The anti-p66 agent need not be, but is optionally formulated with one or more agents that potentiate activity, or that otherwise increase the therapeutic effect. These are generally used in the same dosages and with administration routes as used hereinbefore or about from 1 to 99% of the heretofore employed dosages.

An anti-p66 agent is often administered as a pharmaceutical composition comprising an active therapeutic agent and another pharmaceutically acceptable excipient. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In still some other embodiments, pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

A carrier may bear the agents in a variety of ways, including covalent bonding either directly or via a linker group, and non-covalent associations. Suitable covalent-bond carriers include proteins such as albumins, peptides, and polysaccharides such as aminodextran, each of which have multiple sites for the attachment of moieties. A carrier may also bear an anti-p66 agent by non-covalent associations, such as non-covalent bonding or by encapsulation. The nature of the carrier can be either soluble or insoluble for purposes of the invention. Those skilled in the art will know of other suitable carriers for binding anti-p66 agents, or will be able to ascertain such, using routine experimentation.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences 16th edition, Osol, A. Ed. (1980).

Carriers and linkers specific for radionuclide agents include radiohalogenated small molecules and chelating compounds. A radionuclide chelate may be formed from chelating compounds that include those containing nitrogen and sulfur atoms as the donor atoms for binding the metal, or metal oxide, radionuclide.

Radiographic moieties for use as imaging moieties in the present invention include compounds and chelates with relatively large atoms, such as gold, iridium, technetium, barium, thallium, iodine, and their isotopes. It is preferred that less toxic radiographic imaging moieties, such as iodine or iodine isotopes, be utilized in the methods of the invention. Such moieties may be conjugated to the anti-p66 agent through an acceptable chemical linker or chelation carrier. Positron emitting moieties for use in the present invention include ¹⁸F, which can be easily conjugated by a fluorination reaction with the anti-p66 agent.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the anti-p66 agents can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition.

It will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

Borrelia p66 protein sequence
(SEQ ID NO: 1)
MKSHILYKLI IFLTTSAAIF AADALKEKDI FKINPWMPTF GFENTSEFRL DMDELVPGFE
NKSKITIKLK PFEANPELGK DDPFSAYIKV EDLALKAEGK KGDQFKIDVG DITAQINMYD
FFIKISTMTD FDFNKESLFS FAPMTGFKST YYGFPSNDRA VRGTILARGT SKNIGTIQLG
YKLPKLDLTF AIGGTGTGNR NQENDKDTPY NKTYQGILYG IQATWKPIKN LLDQNEDTKS
VIAETPFELN FGLSGAYGNE TFNNSSITYS LKDKSVVGND LLSPTLSNSA ILASFGAKYK
LGLTKINDKN TYLILQMGTD FGIDPFASDE SIFGHISKAA NFKKETPSDP NKKAEIFDPN
GNALNFSKNT ELGIAFSTGA SIGFAWNKDT GEKESWAIKG SDSYSTRLFG EQDKKSGVAL
GISYGQNLYR SKDTEKRLKT ISENAFQSLN VEISSYEDNK KGIINGLGWI TSIGLYDILR
QKSVENYPTT ISSTTENNQT EQSSTSTKTT TPNLTFEDAM KLGLALYLDY AIPIASISTE
AYVVPYIGAY ILGPSNKLSS DATKIYLKTG LSLEKLIRFT TISLGWDSNN IIELANKNTN
NAAIGSAFLQ FKIAYSGS
Heavy chain variable region of anti-p66 with CDR sequences underlined.
(SEQ ID NO: 2)
QVQLQESGPGLVKPSGTLSLTCAVSGGSISSSNWWSWVRQPPGKGLEWIGEIYHSGSTNYN
PSLKSRVTISVDKSKNQFSLKLSSVTAADTAVYYCARRGNQKDIVVVPAAIFSGENAFDIWGQG
TMVTVSS
(SEQ ID NO: 4, HCDR 1)
SSNWWS
(SEQ ID NO: 5, HCDR 2)
EIYHSGSTNYNPSLKS
(SEQ ID NO: 6, HCDR 3)
RGNQKDIVVVPAAIFSGENAFDI
Light chain variable region of anti-p66 with CDR sequences underlined.
(SEQ ID NO: 3)
VL: DIQMTQSPSSLSASVGDRVTITCQASQDISNYLNWYQQKPGKAPKLLIYDASNLETGVPSR
FSGSGSGTDFTFTISSLQPEDIATYYCQQYDNLPYTFGQGTKLEIK
(SEQ ID NO: 7, LCDR1)
QASQDISNYLN
(SEQ ID NO: 8, LCDR2)
DASNLET
(SEQ ID NO: 9, LCDR3)
QQYDNLPYT

### Sequences

### Example 1

### A CD47 Mimic Made by Borrelia burgdorferi Functions as a Bacterial ‘Don't Eat Me Signal’ to Directly Inhibit Macrophages

Innate immunity, the first line of defense against pathogens, relies on efficient elimination of the invading agents by phagocytes. Thus in the co-evolution of host and pathogen, pathogens developed mechanisms to dampen and evade phagocytic clearance. Here, we report that bacterial pathogens can evade clearance by macrophages through molecular mimicry of a mammalian anti-phagocytic “don't eat me” signal. Using a high affinity structural probe for human CD47, a dominant “don't eat me” signal, we discovered a bacterial protein that mimics CD47's structure on the surface of Borrelia burgdorferi (Bb), a bacterial spirochete that can establish infection in mammals including Lyme Disease (LD). Blockade of the mimic promotes clearance of the infection in vivo. We identified p66, a known virulence factor, as a bacterial mimic of the mammalian “don't eat me” signal CD47. Finally, we determined that patients who return to health following LD infection are more likely to generate antibodies to p66 compared to patients who do not. This study demonstrates molecular mimicry as a means used by Bb to inhibit macrophages and evade phagocytic clearance; this mechanism may have broad implications for understanding host-pathogen interactions and the development of therapeutic strategies to combat bacterial infection.

We recently reported that mammalian cells infected with viruses and bacteria upregulate CD47. Importantly, this expression is a result of a cellular response to the pathogen, though the mechanism by which this occurs remains poorly understood. The poxvirus family is known to conserve a mimic of mammalian CD47, suggesting expression of this protein is functionally important. These mimics have low sequence homology (˜20%) but their structure is tightly conserved. However, of the 25 viruses investigated only 1 mimic from the leporipoxvirus MV, M128L, has been characterized and determined to function in the inhibition of macrophage activation and denoted as a virulence factor.

We hypothesized that, similar to cancer cells and poxviruses, bacteria can also inhibit innate immunity through mimicry. We reasoned that bacterial mimics of the mammalian “don't eat me” signal CD47 may allow bacteria to avoid a macrophage-mediated innate immune response and therefore assist in the establishment and persistence of infection. Macrophages play a key role in preventing and clearing disseminated infections and often form the first line of defense upon infection. The bacterial spirochete Borrelia burgdorferi (Bb) has also been shown to be adept at evading innate immune clearance. Because Bb can establish a persistent infection, we hypothesized that 1) Bb have a mechanism of immune evasion by inhibiting components of the human immune system and 2) Bb express a mimic of the mammalian surface protein CD47.

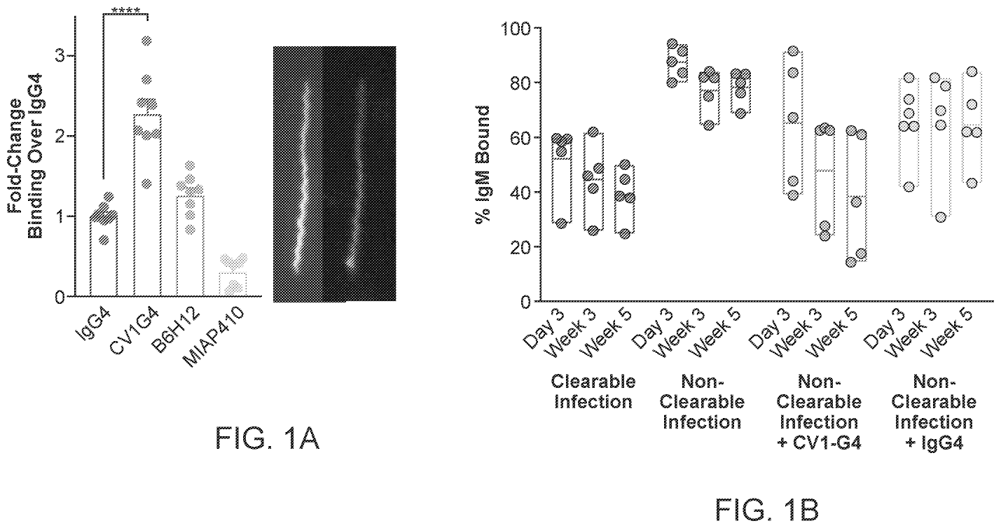

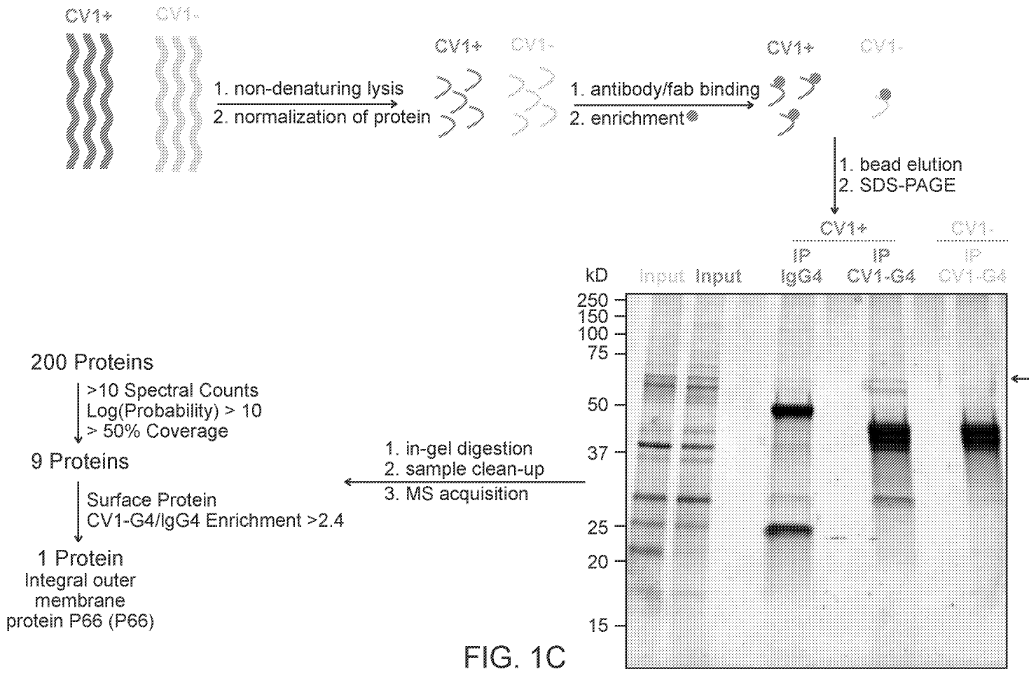

We previously developed a reagent that binds human CD47 with high affinity and blocks its interaction with SIRPα termed CV1. A fusion between CV1 and the human immunoglobulin G4 heavy chain was generated, yielding a divalent, high-affinity CD47-binding reagent termed CV1-G4 that binds human CD47 50,000-fold more strongly than SIRPα. To determine whether Bb displays a protein with structural similarity to the SIRPα binding domain of CD47, we tested whether CV1-G4 as well as commercially-available CD47 antibodies that bind to the surface of Bb, by fluorescence-activated cell sorting (FACS) and microscopy. We determined that CV1-G4 but none of the CD47 antibodies binds to the surface of Bb at 37° C., a temperature physiologically relevant to mammalian host infection (FIG. 1A). CV1-G4 binding was further increased upon a 1 hr incubation with macrophages added into the Bb culture (FIG. 1).

In vivo studies have demonstrated that CV1-G4 treatment promotes macrophage clearance of cancer cells in mouse models. To determine if CV1-G4 treatment would improve clearance of Bb in a mouse infection model, C57BL/6J mice were inoculated with a non-infective dose (10,000) or an infective dose (100,000) of Bb that was pre-treated with either CV1-G4, isotype control or no antibody. Blood was collected at 3 days, 3 weeks or 5 weeks and analyzed for IgM antibodies to determine establishment of infection or clearance.

Having validated the binding of CV1-G4 to the surface of Bb and the functional impact on CV1-G4 treatment on Bb infection, we next wanted to determine the identity of the putative CD47 mimic(s) protein(s) recognized by our high affinity structural probe, CV1-G4. In order to identify the protein(s) in an unbiased manner, we turned to mass spectrometry analysis. First, we performed an immunoprecipitation for enrichment of proteins from Bb cultures using the CV1-G4 reagent or IgG4 as an isotype control. These experiments were performed in cultures known to express the CV1-G4-binding protein or known to be negative for CV1-G4 binding as determined by FACS. Samples were separated by SDS-PAGE and stained with colloidal blue. Gel bands of interest which were significantly enriched in the CV1-G4-positive lysate were excised across all three experimental conditions (CV1-G4-positive CV1-G4 enriched; CV1-G4 positive IgG4 enriched, CV1-G4-negative CV1-G4 enriched), processed and analyzed by mass spectrometry. Nine targets were prioritized, and p66 was identified as the highest-confidence target for CV1-G4 binding taking into consideration cellular localization and quality protein coverage.

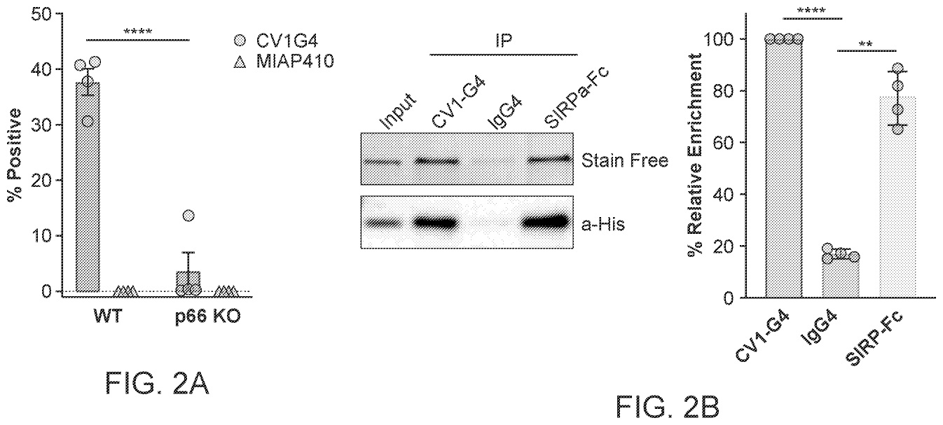

To investigate if P66 is required for CV1-G4 binding on Bb, we compared the percent of CV1-G4 binding to wildtype and p66 KO Bb, to that of MIAP410, a CD47 blockade used in mice that is specific to one epitope on the CD47 protein, via FACS. We validated that the percent of CV1-G4 binding to p66 KO Bb and MIAP410 binding to wildtype and p66 KO Bb were significantly lower than the percent of CV1-G4 binding to wildtype Bb (FIG. 2A). However, CV1-G4 is a matured reagent which has been altered from the original SIRPα sequence to improve affinity to CD47. We wanted to determine if p66 contained similar affinity to the native receptor as it does for the optimized reagent. We used an Fc-fusion of SIRPα in an in vitro binding assay with commercially available recombinant his-tagged p66 (ProspectBio). This assay demonstrated the ability to immunoprecipitate p66 by incubating with CV1-G4 or SIRP-fc, but not by the IgG4 isotype control. p66 bound to affinity reagent was enriched with protein G magnetic beads, digested, and analyzed via SDS PAGE and western blot. The ability to bind p66 with a physiologically relevant receptor demonstrates the ability for this interaction to occur during infection in humans.

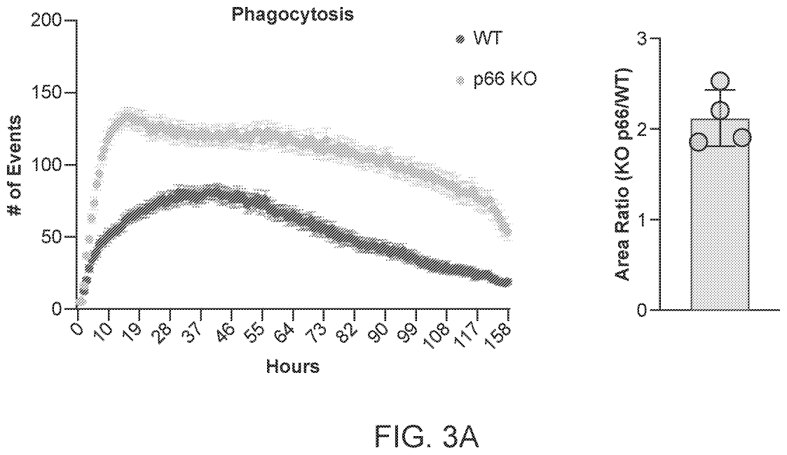

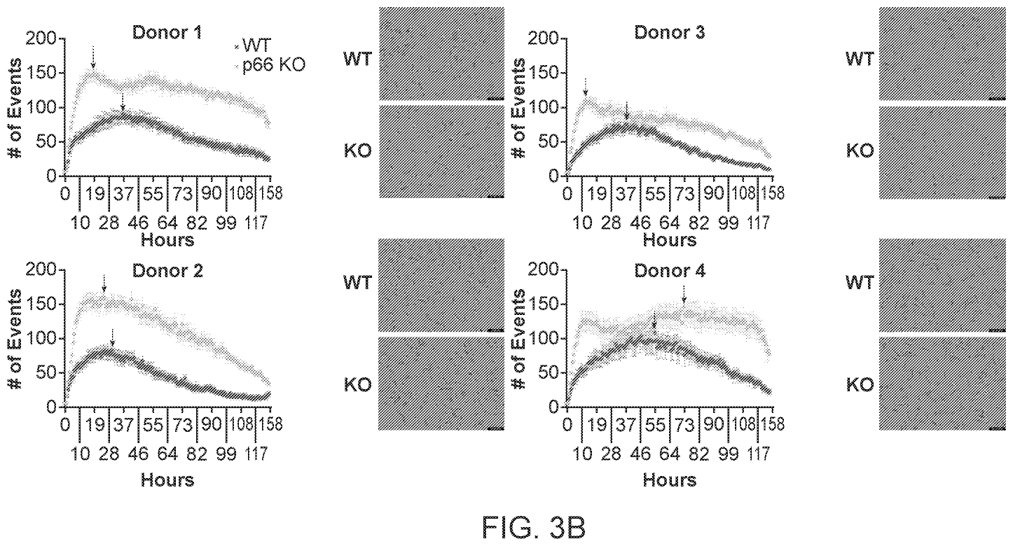

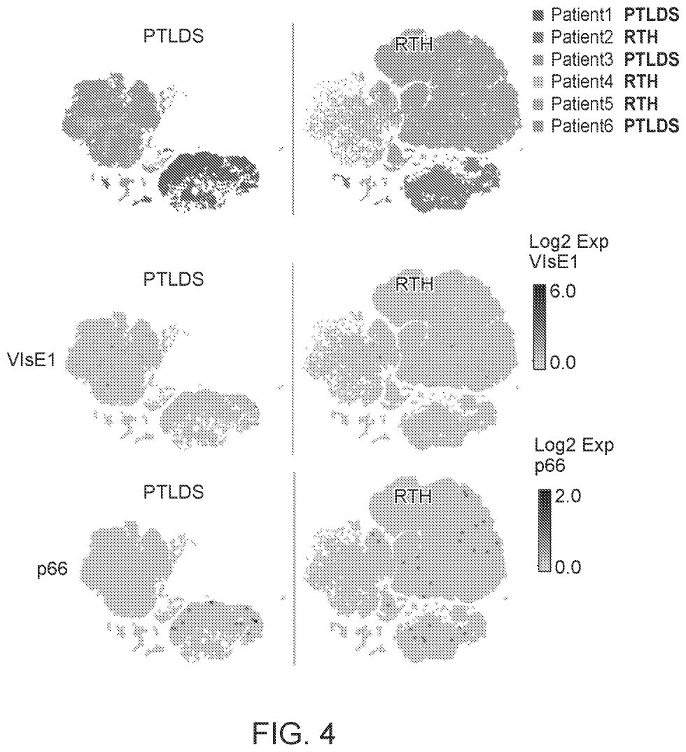

To investigate the role of P66 in mediating macrophage phagocytic clearance of Bb, we performed phagocytosis assays of human monocyte-derived macrophages challenged by pH indicator, pHrodo, labeled wildtype and p66 KO Bb. WT and p66 KO Bb were stained with a pH sensitive rodo (pHrodo) dye to enable imaging of Bb digested within the acidic phagolysosome. The p66 KO Bb on average had higher counts of pHrodo positive phagocytic events over time, compared to that of the wildtype Bb, when challenged against macrophages (FIG. 3, A). Although there is variation in the peak and total number of phagocytosis events between human monocyte-derived macrophages when challenged against p66 KO and wildtype Bb (FIG. 3, B), the ratio of pHrodo positive p66 KO Bb phagocytic events to that of the wildtype Bb was similar across all donors. (FIG. 3, A). Representative IncuCyte images were taken during the peak pHrodo positive wildtype and p66 KO Bb events for each macrophage donor. These images show that, while each donor has a different amount of peak pHrodo positive phagocytosis events, the amount of pHrodo positive p66 KO Bb phagocytosis events are consistently higher for each donor compared to that of the donor's wildtype Bb (FIG. 3, B).

To the best of our knowledge, these findings are the first report of a bacterial mimic of a mammalian “don't eat me” signal whose antigenicity can also be used to clinically differentiate between patients who respond well to antibiotic treatment and patients who do not return to health. Using an affinity-matured reagent CV1-G4, we have demonstrated that the pathogenic spirochete Bb expresses a CD47-like protein on its surface that evades macrophage-mediated phagocytosis. We identified this protein as p66, a protein that has previously been implicated in persistent infection. Chromosomal knockouts of p66 increased Bb clearance and serology studies highlight the clinical importance of developing antibodies against p66.

P66 deficient Bb fail to establish infection. Studies attempting to identify the innate immune cell type antagonized by P66 have found that depletion of either macrophages, or dendritic cells, or neutrophils is insufficient to enable P66 deficient bacteria to establish infection. Intriguingly, all of these cell types can express SIRPα, and this could point to redundancy between different innate immune cell types with phagocytic capacity. It is known that p66 can interact with αvβ3-integrin on the surface of mammalian cells, and this interaction affects gene expression in the host. While p66-mediated integrin binding has been demonstrated to be dispensable for initial infection, it is important for persistence and dissemination in the body. p66 is well conserved across the Borrelia genus, suggesting that other spirochetes may also express their own surface ‘don't eat me’ signals assisting in evasion of macrophage-mediated phagocytosis. The broader applicability of this finding to other bacterial pathogens is of great interest to our laboratory and currently being investigated.

The CD47 protein is upregulated on cancer cells and on cells infected with various pathogens. In addition, CD47 blockade has shown to be a promising therapeutic in regard to cancer. Given the parallels of evasion of immune clearance with CD47 between cancer and infectious disease, we suggest that manipulating this pathway may have therapeutic potential in regard to supporting Bb and other pathogen immune clearance. While immuno oncology has investigated the therapeutic potential of targeting macrophages through immune checkpoint blockade and unleashing their immune-surveillance potential, this blockade can assist in the treatment of pathogenic infections that are notoriously challenging to clear. Traditionally, we have relied on antibiotics for resolving bacterial infections, but these studies suggest that immunomodulatory targets can be therapeutically synergistic.

Given that CV1-G4 is a therapeutic specific for high affinity binding to CD47, there could be improved therapeutic potential for a blockade directly against P66. While a therapeutic against the mimic may perform better than a therapeutic against CD47, CV1-G4 provides the means for identifying other pathogens that manipulate the CD47 pathway and potentially serves as a broad therapeutic that could help clear a range of persistent infections.

### Methods:

Borrelia burgdorferi cultures. In a sterile biosafety cabinet we thawed each stock vial of 1×10⁷ Bb, stored at −80° C., into 50 mL BSK-H complete medium with 6% rabbit serum (Sigma-Aldrich) in a 50 mL conical tube (Fisher Scientific). We used strains Bb GFP, which was generously provided by Jayakumar Rajadas, B31 A3 p66 wildtype and B31 A3 p66 KO C3-14, which were generously provided by Jenifer Coburn. For the p66 KO C3-14 Bb cultures, we selected for p66 KO Bb in 200 ug/mL Kanamycin (Sigma-Aldrich). Each culture was incubated at 37° C. for 7 days, unless otherwise stated.

Primary human donor-derived macrophage generation and stimulation. Leukocyte reduction system chambers from anonymous donors were obtained from the Stanford Blood Center. Peripheral monocytes were purified through successive density gradients using Ficoll (Sigma-Aldrich) and Percoll (GE Healthcare). Monocytes were then differentiated into macrophages by 7-9 days of culture in IMDM with glutamax base+10% AB human serum (Life Technologies).

Fluorescence-Activated Cell Sorting (FACS). FACS was conducted on a BD LSRFortessa at Stanford University School of Medicine FACS Core with BD FACS Diva software. For FACS analysis of Bb species, LSRFortessa cytometor threshold levels were modified to parameter SSC 400. Voltages were set to FSC 300 and SSC 230, collected in log mode.

CV1-G4 and MIAP410 Bb staining. We added 20 μL per well of wildtype and p66 KO Bb cultures to a 96 well v-bottom plate (Corning). After spinning for 10 minutes at 1500 g at 4° C. and aspirating the supernatant, we resuspended the Bb in 30 μL per well of either CV1-G4 or MIAP410 at 10 ug/mL and let the plate of Bb stain on ice for 30 minutes, protected from light. After staining, we washed the plate twice with PBS by topping up each well with PBS to a volume of 150 uL, spinning the plate for 10 minutes at 1500 g at 4° C., and aspirating the supernatant (every wash after this was done in the same way). After the second wash, we resuspended the Bb in 30 uL per well of 1:200 Alexa Fluor 647 anti-human IgG (Jackson ImmunoResearch) or Alexa Fluor 647 anti-mouse IgG (Jackson ImmunoResearch) in PBS. We had the plate incubate on ice for 20 minutes, protected from light. After washing twice more with PBS, we fixed the Bb in 100 uL per well of 4% paraformaldehyde (EMS) for 10 minutes at room temperature, protected from light. We washed the plate twice more with PBS and resuspended the Bb in 200 uL per well of FACS buffer (500 mL PBS, 2% fetal bovine serum, and 1 mmol EDTA (Thermo Fisher Scientific)). We kept the plate protected from light until running the plate on a BD Fortessa, gating our Bb for FSC, SSC, as well as positive and negative Alexa Fluor 647 fluorescence.

Time-lapse live-cell-microscopy-based phagocytosis assay. Human serum derived macrophages were lifted from the plates using 5 mL of TrypLE (Thermo Fisher Scientific) per plate. Once lifted, the macrophages in TrypLE were diluted with 5 mL of PBS and transferred to a 15 mL conical tube (Fisher Scientific). The macrophages were counted using Trypan Blue (Life Technologies) and spun down at 300 g for 5 min at 4° C. After aspirating the supernatant, the macrophages were resuspended in R10 without phenol red at a concentration of 1.5×10⁵ cells/mL. 100 uL of the macrophage solution was plated in each well of 96-well Imagelock plates (Essen) for a total of 150,000 macrophages per well. The plate with macrophages was left to incubate at 37° C. for 30 minutes to ensure macrophage adherence to the plate.

P66 KO and wildtype 7 day Bb cultures in 50 mL conical tubes were spun down at 1500 g for 10 min at 4° C. and resuspended into 10 mL of 1:20,000 pHrodo (Essen) in PBS. After 1 hour of incubation at 37° C., 1 mL of fetal bovine serum was added to each Bb solution. They were spun down at 1500 g for 10 min at 4° C. and resuspended in R10 without phenol red at a concentration of 3×10⁶ Bb/mL. After the macrophages have adhered to the plate, 50 uL of the Bb solution was plated in each well of 96-well Imagelock plates for a total of 1,500,000 Bb per well. 100 uL of R10 without phenol red was added to wells without macrophages and 50 uL of R10 without phenol red was added to wells without Bb.

Phagocytosis assay plates were then placed in an incubator at 37° C. and imaged at 45-intervals for 6.57 days using an IncuCyte (Essen). The first image time point (reported as t=0) was generally acquired within 30 min of co-culture. Images were acquired using a 20×objective at 800-ms exposures per field. Phagocytosis events were calculated as the number of pHrodo-red+events per well and values were normalized to the maximum number of events measured across technical replicates per donor.

CV1-G4 versus IgG4. GFP Bb cultures in 50 mL conical tubes were spun down at 1500 g for 10 min at 4° C. and counted. Cultures were resuspended into BSK at 1×10⁶ Bb/mL with 10 ug/mL of either CV1-G4, IgG4, or no antibody. After incubating for 1 hour at 37° C., the cultures were washed with PBS and resuspended into BSK at a concentration of either 5×10⁴ or 5×10⁵ Bb/mL. We injected each mouse with 200 uL of Bb solution for either an infection of 1×10⁴ or 1×10⁶ Bb per mouse. Retro-orbital bleeds were performed on days 3, 21, and 35 for each mouse. The blood was collected into 10 uL EDTA and spun down at 1500 RPM for 5 min at 4° C. The samples were then spun down at 10,000 g for 10 min at 4° C., in order to remove the platelets, and the plasma without platelets was collected from the samples and stored at −80° C. The plasma without platelets was added to Bb in a 96-well plate (Corning). Using an Anti-Mouse IgM (BioLegend), we characterized the amount of IgM present on Bb with infected mouse serum via FACS.

Immunoprecipitation, strain-free imaging, and blot of recombinant P66. Recombinant his-tagged P66 was purchased from ProspectBio in a solution of 20 mM HEPES. P66 was diluted using buffer A (20 mM HEPES, pH 7.5 in MilliQ water) to a concentration of 500 ng of protein per sample. Samples received 5 ng of affinity reagent and were then rotated at 4° C. for 1.5 hrs. Dynabeads™ Protein G magnetic affinity beads were prepared with 3×200 μL washes of buffer A. 2 uL of beads were used per 500 ng of protein. Washed beads were suspended in 10× volume of buffer A (20 μL) to ease transfer to samples. Beads were incubated with affinity reagent labeled P66 at 4° C. for 1.5 hrs. Samples were rinsed 3× with buffer A before resuspension in 10 μL 2×SDS Loading buffer with 350 mM BME and 50 μL buffer A. Samples were heated for 5 mins. at 95° C. to denature peptides from beads. All 150 μL of sample was loaded into a TGX Stain Free Precast Gels (Biorad) with 1×Tris/Gly buffer and run at 200V for approximately 45 mins. The polyacrylamide gel was then imaged stain free on a CHEMIDOC (Biorad). Gel was incubated overnight at 4° C. in transfer buffer (50 mM Tris, 40 mM Glycine, 0.1% SDS, 20% MeOH).

The following day, protein was transferred from gel onto blotting membrane. Blot was blocked with 5% milk in 1×TBST for 1 hr., rocking at RT. Blot membrane was then incubated with 1:1000 dilution anti-his conjugated to HRP (Thermo Scientific) in 5% milk in 1×TBST for 1 hr., rocking at RT. Blot membrane was then washed 3× with 1×TBST, rocking for 5 min. during each wash cycle. Following washes, Radiance Plus ECL (Azure Biosystems) chemiluminescent substrate and activating hydrogen peroxide were incubated with blot for approximately 5 minutes. Resulting protein bands were visualized using the chemiluminescent setting on CHEMIDOC (Biorad).

In Vivo. All experiments were carried out in accordance with ethical care guidelines set by the Stanford University Administrative Panel on Laboratory Animal Care (APLAC). In compliance with Stanford APLAC protocol (30109), Borrelia burgdorferi infected mice were housed in Stanford University's core research animal facility (RAF). Female mice were used for all studies. Investigators were not blinded for animal studies.

### Example 2

### p66 Blockade as a Therapeutic Strategy for the Treatment of Lyme Disease

Bacterial evasion of immune clearance is a major roadblock in treating pathogenic infection. We have found that the bacteria responsible for Lyme Disease (LD) evades the innate immune system through mimicry of a mammalian protein, CD47. CD47, which we have previously discovered to be a ‘don't eat me’ signal, blocks the ability of immune cells such as macrophages and neutrophils to engulf and destroy cells, including cancer cells. Antibody blockade of CD47 has been shown to be an effective treatment in many types of cancer. An antibody specific to the bacterial mimic of CD47, called p66, can be a viable treatment strategy for LD.

As disclosed in Example 1, bacterial pathogens can evade clearance by phagocytes through molecular mimicry of a mammalian anti-phagocytic “don't eat me” signal. Using our engineered CV1G4 as a high affinity antibody for CD47, a dominant “don't eat me” signal, we discovered a bacterial protein that mimics CD47's structure on the surface of Borrelia burgdorferi (Bb), a bacterial spirochete that established Lyme Disease (LD) infection in mammals (FIG. 1A). Blockade of the mimic promotes clearance of the infection in vivo (FIG. 1B). We identified p66, a known virulence factor, as a bacterial mimic of the mammalian “don't eat me” signal CD47 (FIG. 1C).

We determined that patients who return to health (RTH) following LD infection are more likely to generate antibodies to p66 compared to patients who do not (FIG. 2A). Using uniquely barcoded antigens and 10×Genomics technology to sequence antigen-specific B cells from Post-Treatment Lyme Disease Syndrome (PTLDS) and RTH individuals (n=3 each), we observed that individuals treated for Lyme disease and that have returned to health have elevated responses to p66 when compared to individuals that suffer from PTLDS (FIG. 2A). To validate these data, p66-binding sequences from RTH individuals, NSR32 and NSR33, were recombinantly expressed in human IgG1 format and tested for binding to p66 and other Lyme antigens. Of these, NSR32 bound p66, but was observed to bind other Lyme antigens as well (FIG. 2B). This promiscuity may be explained by the fact that the sequence was derived from an IgM B-Cell Receptor (BCR). Little to no binding on the part of NSR33 could be due to the extremely low affinity of this sequence. Despite this, these sequences provide a starting point to develop an antibody with improved specificity and affinity to p66.

These findings indicate molecular mimicry as a means used by Bb to inhibit macrophages and evade phagocytic clearance; this mechanism may have broad implications for understanding host-pathogen interactions and the development of therapeutic strategies to combat bacterial infection.

Develop a high-affinity p66 antibody. Patients who have recovered from Lyme disease and returned to health make robust anti-p66 antibody responses. The sequences identified as anti-p66 antibodies are affinity matured to generate a high affinity therapeutic reagent. This is then combined with appropriate human or mouse FC components and tested in the respective models for eliciting maximum immune clearance.

B-cells from additional RTH individuals (n=10) are sequences with barcoded antigen technology to identify additional p66-binding BCRs. These are recombinantly expressed in human IgG1 format, to validate binding to p66 by ELISAs. Sequences that bind p66 preferentially are identified for affinity maturation.

In order to affinity mature p66 antibodies, mutagenesis approaches such as error-prone PCR are used to mutate mainly the Complementarity-Determining Regions (CDRs) of the antibody in scFv format, before displaying the sequences on the surface of phage and performing rounds of positive selection against p66, while simultaneously eliminating sequences that bind human CD47 (to avoid an autoimmune reaction). Antibody affinities are determined by Bio-layer interferometry.

The scFv sequences of interest are recombinantly expressed as human IgG1 rMabs. These rMabs are validated by Bio-layer interferometry for affinity and by ELISA for specificity. The human versions of the best blocking rMabs are combined with Fc regions of IgG1 or IgM for testing Bb clearance by human macrophages in vitro. IgM is pentavalent and has high avidity, and a majority of the p66 binding sequences thus far are derived from IgM BCRs. Therefore, this Fc format is included in our assays to test efficacy. Any sequences that bind human CD47 are avoided, thus eliminating the need to test the IgG4 Fc format in our assays. The mouse versions of the best blocking rMabs (IgG2a or IgM format) are tested in vivo in a mouse infection model for ability to enhance Bb-GFP clearance and/or prevent infection from establishing.

Determine therapeutic impact of p66 blockade on Bb clearance. The highest affinity p66 antibodies are used in comparison to CV1G4 to test clearance efficacy of Bb by human macrophages and neutrophils in vitro. These therapeutic reagents are tested in an in vivo infection model to evaluate their prophylactic and therapeutic potential.

To determine the effect that p66 blockade has in mediating phagocytic clearance of Bb by macrophages and neutrophils, phagocytosis assays of human monocyte-derived macrophages and peripheral blood neutrophils challenged with Bb are conducted. WT or p66 KO Bb are stained with a pH sensitive rodo (pHrodo) dye to enable imaging of Bb digested within the acidic phagolysosome. The highest affinity anti-p66 antibodies are tested through IncuCyte analysis, measuring red-fluorescence counts over time. Red fluorescence event counts are generated from pHrodo-labeled Bb that have entered the acidic lysosome of macrophages or neutrophils for degradation. Fc variation on the anti-p66 antibodies (IgM vs IgG1) is compared for efficacy and benchmarked against CV1G4.

To investigate the therapeutic efficacy of p66 blockade in vivo a mouse model of Bb infection is used. More specifically, mice intraperitoneally are injected with a dose of Bb that is unclearable without treatment, testing both prophylactic (pre-infection) and therapeutic (post-infection) regimens of treatment with high affinity anti-p66 antibodies with either murine IgG2a or IgM Fc domains. These are compared to antibody isotype controls and CV1G4 treatment. To determine infection progression, we track the ankle swelling through caliper measurements, Bb bacterial load by flow cytometry, and the immune response by profiling anti-Bb IgM and IgG responses over time. It is expected that mice infected with Bb and treated with p66 affinity reagent will achieve reduced swelling, more-rapid bacterial clearance, and a lowered IgM response over time compared to isotype controls.

The domains of p66 required for interaction with SIRPα that function as a ‘don't eat me’ signal are identified. p66 consists of 7 domains. The extracellular integrin binding and/or the surface loop domains may be responsible for recognition. His-tagged p66 is readily expressed in E. coli and still retains SIRPα binding (FIG. 3A). p66 domain deletion mutants are recombinantly expressed and purified using similar methods. Using p66 mutants, an in vitro binding assay with IgG4, CV1-G4 and SIRPα-Fc (FIG. 3B) is employed. For domain deletions that lose enrichment with SIRPα-Fc and CV1-G4 compared to full-length, domain(s) are expressed on their own and resubmitted to the workflow for verification of the domain's interactions. This will clarify how p66 mimics the binding interaction of CD47 with SIRPα and inform the maturation of a high-affinity antibody reagent against p66 for the development of a therapeutic. Experiments are performed at least in triplicate and relative enrichment compared to Full-Length CV1-G4 enrichment. Statistical analysis is conducted by paired t-test.

It is investigated by flow cytometry and mass spectrometry binding assay if other spirochetes also express a CD47 mimic as a means by which to evade immune clearance (FIGS. 3C and 3D). B. hermsii and B. duttonii are predicted to have a p66 protein with high sequence homology to that of Bb. Bacteria including Borrelia hermsii, Borrelia duttonii, Borrelia miyamotoi and Borrelia turicatae are stained with CV1-G4, SIRPα-Fc, a p66 affinity matured antibody or isotype control and subjected to flow cytometry analysis. Experiments are performed at least 3 independent times with each condition performed in triplicate. Bacteria that show labeling above isotype control/background are further evaluated using our in vitro binding assay that previously identified Bb p66 as a CD47 mimic. More specifically, bacteria of interest are lysed under non-denaturing conditions and proteins binding to CV1-G4, SIRPα-Fc, a p66 affinity matured antibody or isotype control is purified prior to sample preparation for MS analysis.

### CITATIONS

(1) Majeti, R.; Chao, M. P.; Alizadeh, A. A.; Pang, W. W.; Jaiswal, S.; Gibbs, K. D.; van Rooijen, N.; Weissman, I. L. CD47 Is an Adverse Prognostic Factor and Therapeutic Antibody Target on Human Acute Myeloid Leukemia Stem Cells. Cell 2009, 138 (2), 286-299. https://doi.org/10.1016/j.cell.2009.05.045.
(2) Barkal, A. A.; Brewer, R. E.; Markovic, M.; Kowarsky, M.; Barkal, S. A.; Zaro, B. W.; Krishnan, V.; Hatakeyama, J.; Dorigo, O.; Barkal, L. J.; Weissman, I. L. CD24 Signalling through Macrophage Siglec-10 Is a Target for Cancer Immunotherapy. Nature 2019, 572 (7769), 392-396. https://doi.org/10.1038/s41586-019-1456-0.
(3) Gordon, S. R.; Maute, R. L.; Dulken, B. W.; Flutter, G.; George, B. M.; McCracken, M. N.; Gupta, R.; Tsai, J. M.; Sinha, R.; Corey, D.; Ring, A. M.; Connolly, A. J.; Weissman, I. L. PD-1 Expression by Tumour-Associated Macrophages Inhibits Phagocytosis and Tumour Immunity. Nature 2017, 545 (7655), 495-499. https://doi.org/10.1038/nature22396.
(4) Barkal, A. A.; Weiskopf, K.; Kao, K. S.; Gordon, S. R.; Rosental, B.; Yiu, Y. Y.; George, B. M.; Markovic, M.; Ring, N. G.; Tsai, J. M.; McKenna, K. M.; Ho, P. Y.; Cheng, R. Z.; Chen, J. Y.; Barkal, L. J.; Ring, A. M.; Weissman, I. L.; Maute, R. L. Engagement of MHC Class 1 by the Inhibitory Receptor LILRB1 Suppresses Macrophages and Is a Target of Cancer Immunotherapy. Nat Immunol 2018, 19 (1), 76-84. https://doi.org/10.1038/s41590-017-0004-z.
(5) Liu, B.; Guo, H.; Xu, J.; Qin, T.; Guo, Q.; Gu, N.; Zhang, D.; Qian, W.; Dai, J.; Hou, S.; Wang, H.; Guo, Y. Elimination of Tumor by CD47/PD-L1 Dual-Targeting Fusion Protein That Engages Innate and Adaptive Immune Responses. mAbs 2018, 10 (2), 315-324. https://doi.org/10.1080/19420862.2017.1409319.
(6) Barclay, A. N.; Hatherley, D. The Counterbalance Theory for Evolution and Function of Paired Receptors. Immunity 2008, 29 (5), 675-678. https://doi.org/10.1016/j.immuni.2008.10.004.
(7) Tal, M. C.; Torrez Dulgeroff, L. B.; Myers, L.; Cham, L. B.; Mayer-Barber, K. D.; Bohrer, A. C.; Castro, E.; Yiu, Y. Y.; Lopez Angel, C.; Pham, E.; Carmody, A. B.; Messer, R. J.; Gars, E.; Kortmann, J.; Markovic, M.; Hasenkrug, M.; Peterson, K. E.; Winkler, C. W.; Woods, T. A.; Hansen, P.; Galloway, S.; Wagh, D.; Fram, B. J.; Nguyen, T.; Corey, D.; Kalluru, R. S.; Banaei, N.; Rajadas, J.; Monack, D. M.; Ahmed, A.; Sahoo, D.; Davis, M. M.; Glenn, J. S.; Adomati, T.; Lang, K. S.; Weissman, I. L.; Hasenkrug, K. J. Upregulation of CD47 Is a Host Checkpoint Response to Pathogen Recognition. mBio 2020, 11 (3), e01293-20, /mbio/11/3/mBio.01293-https://doi.org/10.1128/mBio.01293-20.
(8) Farré, D.; Martínez-Vicente, P.; Engel, P.; Angulo, A. Immunoglobulin Superfamily Members Encoded by Viruses and Their Multiple Roles in Immune Evasion. Eur. J. Immunol. 2017, 47 (5), 780-796. https://doi.org/10.1002/eji.201746984.
(9) Cameron, C. M.; Barrett, J. W.; Mann, M.; Lucas, A.; McFadden, G. Myxoma Virus M128L Is Expressed as a Cell Surface CD47-like Virulence Factor That Contributes to the Downregulation of Macrophage Activation in Vivo. Virology 2005, 337 (1), 55-67. https://doi.org/10.1016/j.virol.2005.03.037.
(10) Weiskopf, K.; Ring, A. M.; Ho, C. C. M.; Volkmer, J.-P.; Levin, A. M.; Volkmer, A. K.; Özkan, E.; Fernhoff, N. B.; van de Rijn, M.; Weissman, I. L.; Garcia, K. C. Engineered SIRPα Variants as Immunotherapeutic Adjuvants to Anticancer Antibodies. Science 2013, 341 (6141), 88-91. https://doi.org/10.1126/science.1238856.
(11) Curtis, M. W.; Hahn, B. L.; Zhang, K.; Li, C.; Robinson, R. T.; Coburn, J. Characterization of Stress and Innate Immunity Resistance of Wild-Type and Δp66 Borrelia Burgdorferi. Infect Immun 2017, 86 (2), e00186-17. https://doi.org/10.1128/IA1.00186-17.
(12) Bárcena-Uribarri, I.; Thein, M.; Sacher, A.; Bunikis, I.; Bonde, M.; Bergström, S.; Benz, R. p66 Porins Are Present in Both Lyme Disease and Relapsing Fever Spirochetes: A Comparison of the Biophysical Properties of p66 Porins from Six Borrelia Species. Biochimica et Biophysica Acta (BBA)—Biomembranes 2010, 1798 (6), 1197-1203. https://doi.org/10.1016/j.bbamem.2010.02.011.

1. A method of inhibiting or treating an infection by a Borrelia pathogen, the method comprising administering an effective amount of an anti-p66 agent that reduces binding of p66 on the pathogen to signal regulatory protein □(SIRP□) on a phagocytic cell, where the agent (i) specifically binds to p66 (SEQ ID NO:1), or (ii) is a p66 polypeptide or derivative thereof, wherein the effective amount of the agent is sufficient to increase phagocytosis of a Borrelia pathogen by a phagocytic cell.

2. (canceled)

3. The method of claim 1, wherein the phagocytic cell is a macrophage.

4. The method of claim 1, wherein the agent specifically binds to p66.

5. The method of claim 4, wherein the agent is an antibody.

6. The method of claim 5, wherein the antibody is chimeric, humanized or human.

7. The method of claim 5, wherein the antibody is affinity matured.

8. The method of claim 5 wherein the antibody comprises an Fc domain selected from the group consisting of IgG1, IgG2, IgG3, IgG4, IgA, IgE, IgD, and IgM.

9. (canceled)

10. The method of claim 5, wherein the antibody comprises as a variable region binding sequence the CDR sequences of SEQ ID NO:2 and SEQ ID NO:3, or a variant thereof.

11. The method of claim 10, wherein the variant is affinity matured in vitro.

12. The method of claim 5, wherein the antibody comprises as a variable region binding sequence SEQ ID NO:2 and SEQ ID NO:3, or a variant thereof.

13. The method of claim 1, wherein the Borrelia pathogen is Borrelia burgdorferi.

14. The method of claim 1, wherein the agent is a p66 polypeptide or derivative thereof.

15. The method of claim 14, wherein the polypeptide comprises at least a portion of the sequence of SEQ ID NO:1.

16. The method of claim 14, wherein the p66 polypeptide is a soluble fragment of SEQ ID NO:1 or a derivative thereof.

17. The method of claim 14 wherein the p66 polypeptide is an affinity matured variant of p66.

18. The method of claim 14, wherein the p66 polypeptide is fused to an antibody Fc domain.

19. The method of claim 18, wherein the antibody is selected from the group consisting of IgG1, IgG2, IgG3, IgG4, IgA, IgE, IgD, and IgM.

20. The method of claim 1, wherein the method is performed in vivo or in vitro.

21. The method of claim 1, wherein the Borrelia pathogen is Borrelia burgdorferi.

22. A composition comprising an anti-p66 agent, where the agent (i) specifically binds to p66 (SEQ ID NO:1), or (ii) is a p66 polypeptide or derivative thereof according to claim 1.
